(19) United States
(12) Patent Application Publication
(10) Pub. No.: US 2026/0115249 A1
(43) Pub. Date: Apr. 30, 2026

(54) NEURAL REPAIR COMPOSITION, METHOD OF PREPARATION, AND USE THEREOF

(71) Applicant: SHENZHEN JINFENG BIOMEDICAL TECH. CO., LTD., SHENZHEN (CN)

(72) Inventors: BING ZHOU, SHENZHEN (CN); XINGYU HE, SHENZHEN (CN); YONGMEI XIE, SHENZHEN (CN)

(21) Appl. No.: 19/157,990

(22) PCT Filed: May 14, 2024
(86) PCT No.: PCT/CN2024/093120

§ 371 Date: Aug. 19, 2025

(30) Foreign Application Priority Data
Jul. 20, 2023 (CN) 202310895575.3

(51) Int. Cl. A61K 36/9066; A61K 31/121; A61K 31/201; A61K 31/202; A61K 31/355; A61K 31/4415; A61K 31/7084; A61K 36/77; A61P 25/16; A61P 25/24; A61P 25/28; A61P 39/06

(52) U.S. Cl. CPC A61K 36/9066; A61K 31/121; A61K 31/201; A61K 31/202; A61K 31/355; A61K 31/4415; A61K 31/7084; A61K 36/77; A61P 25/16; A61P 25/24; A61P 25/28; A61P 39/06; A61K 2236/333; A61K 2236/51

(57) ABSTRACT

A neural repair composition, and a preparation method and use thereof are provided. The neural repair composition includes the following components in parts by weight: 0.1 part to 10 parts of curcumin or a turmeric powder and 4 parts to 460 parts of an Acer truncatum bunge seed oil. Through the synergistic interaction between Acer truncatum bunge seed oil and curcumin, the composition demonstrates excellent anti-aging and neural repair efficacy, while exhibiting mild action and non-irritating properties to the human body. The composition shows significant effects in preventing and treating neurodegenerative diseases, particularly in alleviating depression and enhancing memory.

### CROSS REFERENCE TO RELATED APPLICATION

The present application is a U.S. national phase PCT application No. PCT/CN2024/093120 filed on May 14, 2024, which claims priority to Chinese Patent Application No. CN202310895575.3 filed to the China National Intellectual Property Administration (CNIPA) on Jul. 20, 2023 and entitled “NEURAL REPAIR COMPOSITION. METHOD OF PREPARATION. AND USE THEREOF”, both of which are incorporated herein by reference in their entirety.

### TECHNICAL FIELD

The present disclosure relates to the technical field of medicines and health products, and in particular to a neural repair composition, a method of preparation, and use thereof.

### BACKGROUND

In modem society, accelerated lifestyles, intense competition, and high stress levels, combined with various unhealthy habits, keep the brain and nervous system in prolonged states of tension and fatigue, namely a suboptimal health condition. This predisposes the human nervous system to damage, accelerates organism aging, and increases susceptibility to neurodegenerative diseases. Neurodegenerative diseases, characterized by neuronal dysfunction or death, include Alzheimer's disease, Parkinson's disease, amyotrophic lateral sclerosis (ALS), and the like. Their etiology involves genetic, environmental, and metabolic factors, yet no effective therapies currently exist, imposing significant physical, emotional, and economic burdens on patients and families.

Acer truncatum bunge seed oil, a vegetable oil extracted from the seeds of Acer truncatum bunge, contains bioactive components such as unsaturated fatty acids, vitamin E, and phytosterols, demonstrating antioxidant, anti-inflammatory, and lipid-lowering properties. Curcumin, a polyphenolic compound derived from turmeric rhizomes, exhibits effects including antioxidation and anti-inflammation, and inhibition of amyloid protein deposition, among others. While numerous anti-aging skincare and health products with antioxidant effects have emerged, many commercially available formulations rely heavily on synthetic antioxidants to enhance efficacy. These additives may cause skin irritation and compromise neural repair outcomes. Although efforts persist to develop natural antioxidant products with superior performance, existing solutions still fall short in delivering optimal antioxidant and anti-aging effects.

As a result, there is an urgent need to develop a novel health product that synergistically combines enhanced anti-aging benefits with improved neural repair efficacy.

### SUMMARY

An objective of the present disclosure is to overcome the limitations of existing anti-aging products (particularly those targeting neurodegenerative disease prevention) which exhibit significant irritancy and insufficient anti-aging performance. The present disclosure provides a novel healthcare composition with superior anti-aging and neural repair efficacy. Through synergistic interaction among Acer truncatum bunge seed oil, curcumin, and phosphatidylserine (PS), the composition demonstrates excellent anti-aging and neural repair efficacy, while exhibiting mild action and non-irritating properties to the human body.

Another objective of the present disclosure is to provide a method for preparing the neural repair composition.

A further objective of the present disclosure is to provide use of the neural repair composition in preparation of a health product for preventing or treating a neurodegenerative disease.

To achieve the above objectives, the present application adopts the following technical solutions:

The present disclosure provides a neural repair composition, including the following components in parts by weight:

0.1 part to 10 parts of curcumin or a turmeric powder and 4 parts to 460 parts of an Acer truncatum bunge seed oil.

Curcumin or turmeric powder (containing the curcumin), a polyphenolic compound derived from turmeric rhizomes, exhibits effects including antioxidation and anti-inflammation, inhibition of amyloid protein deposition, among others. The curcumin or turmeric powder used in the present disclosure may be commercially available products or obtained by extraction/preparation from rhizomes of Zingiberaceae plants. The curcumin or turmeric powder may be prepared by a method including the following steps:

Adding a dried turmeric root powder to ethanol for ultrasonic extraction to obtain an ethanol extract, which yields curcumin; concentrating the ethanol extract under vacuum to obtain a concentrate; and freeze-drying the concentrate to obtain the turmeric powder.

In some embodiments, the ethanol has a volume fraction of 70% to 95%; the ultrasonic extraction is performed at 40° C. to 60° C. for 30-60 min; the vacuum concentration is performed at 40° C. to 60° C. under a pressure of 0.05-0.1 MPa; the freeze-drying is performed at −40° C. to −20° C.

The curcumin or turmeric powder includes 2-5 wt % of curcuminoid, where the curcuminoid includes: 50-60 wt % of C1 curcumin (Curcumin), 15-17 wt % of C2 demethoxycurcumin (Demethoxycurcumin), and 0.5-1.5 wt % of C3 bisdemethoxycurcumin (Bisdemethoxycurcumin).

Acer truncatum bunge seed oil, a vegetable oil extracted from the seeds of Acer truncatum bunge, is rich in bioactive components such as unsaturated fatty acids, vitamin E, and phytosterols. Recognized globally by the scientific community as a dual-functional agent, this component uniquely repairs damaged neural pathways, regenerates nerve fibers, and promotes neuronal cell regeneration. Deficiency of this oil has been linked to neurological disorders including post-stroke sequelae, Alzheimer's disease, cerebral palsy, brain atrophy, memory decline, insomnia, and cognitive impairment. Additionally, it exhibits antioxidant, anti-inflammatory, and lipid-lowering properties.

In the present disclosure, the Acer truncatum bunge seed oil may be commercially available products or extracted/prepared from Acer truncatum bunge kernels. The Acer truncatum bunge seed oil includes the following relative mass contents: linoleic acid: greater than 30%, arachidonic acid: greater than 12%, and nervonic acid: greater than 4%.

In some embodiments, the Acer truncatum bunge seed oil includes 40 wt % to 50 wt % of linoleic acid, 15 wt % to 20 wt % of arachidonic acid, and 5 wt % to 8 wt % of nervonic acid.

In some embodiments, the neural repair composition includes the following components in parts by weight:

1 part to 5 parts of the curcumin or the turmeric powder and 45 parts to 250 parts of the Acer truncatum bunge seed oil.

Within this preferred range, the composition exhibits enhanced anti-aging efficacy and superior neural repair performance.

The inventors have discovered through extensive research that the synergistic interaction between curcumin and Acer truncatum bunge seed oil significantly enhances their intrinsic antioxidant capacity and amplifies neural repair mechanisms. This dual action effectively prevents and mitigates neurodegenerative diseases such as Alzheimer's disease, Parkinson's disease, and ALS.

The PS is preferably flavin adenine dinucleotide (FAD). As a flavin coenzyme prosthetic group derived from vitamin B₂, FAD participates in multiple redox reactions in the human body, primarily facilitating oxidative dehydrogenation of organic compounds such as fatty acids. Additionally, PS regulates metabolism by interacting with fatty acids generated during physiological reactions, enhances vitamin absorption, improves immune function, and amplifies antioxidant and neural repair efficacy. Notably, the inventors have creatively discovered that the combined action of PS with curcumin and Acer truncatum bunge seed oil significantly improves symptoms in depression patients and enhances memory restoration.

To further optimize antioxidant and neural repair effects, 0.01 part to 2 parts of a vitamin may be added to the neural repair composition, including: 0.01 part to 1 part of vitamin E and 0.01 part to 1 part of vitamin B₆. The vitamin E is selected from the group consisting of natural or synthetic α-tocopherol and a derivative thereof.

In some embodiments, the excipient is pharmaceutically acceptable and includes, but is not limited to, at least one of a diluent, a preservative, and a suspending agent. Diluent may be: polyethylene glycol, propylene glycol, polysorbate, glycerol, peanut oil, corn oil, soybean oil, or combinations thereof. Preservative may be: benzoic acid, sorbic acid, chlorobutanol, benzyl alcohol, methylparaben, or combinations thereof. Suspending agent may be: beeswax, hydrogenated vegetable oil, hydrogenated castor oil, acacia gum, soybean lecithin, cocoa butter, propylene glycol monocaprylate, glyceryl monocaprylate, carboxymethylcellulose, hydroxypropyl cellulose, hydroxyethyl methylcellulose, or combinations thereof.

The present disclosure further provides a method for preparing the neural repair composition, including the following steps:

Thoroughly mixing the components in the parts by weight to obtain the neural repair composition.

The disclosure also encompasses use of the anti-aging composition in manufacturing health products for preventing and treating a neurodegenerative disease. The neurodegenerative disease includes but is not limited to Alzheimer's disease, Parkinson's disease, ALS, depression, and memory impairment.

Compared with the prior art, embodiments of the present disclosure has the following beneficial effects:
1) Prolonging survival of ALS (also known as Lou Gehrig's disease) patients, preventing and delaying neurodegenerative diseases. ALS is a fatal neuromuscular disorder causing progressive degeneration and death of motor neurons, with currently no effective treatment available. The composition of the present disclosure may protect motor neurons from oxidative stress and inflammatory damage through nervonic acid and curcumin, delay neuronal functional decline, and improve quality of life for ALS patients.
2) Reducing Aβ protein (namely β-amyloid) aggregation in AD (namely Alzheimer's disease) patients and alleviating cognitive impairment. AD is a common senile dementia characterized by memory loss, language dysfunction, and impaired judgment. The main pathological feature of AD involves senile plaques formed by Aβ protein deposits in the brain, leading to neuronal death and synaptic dysfunction. The composition of the present disclosure may inhibit Aβ protein production and aggregation through nervonic acid and curcumin, reduce plaque formation, protect neuronal and synaptic structure/function, and improve cognitive abilities in AD patients.
3) Alleviating depression and anxiety symptoms while improving emotional states. Depression and anxiety are common mental disorders manifesting as low mood, restlessness, loss of interest, and self-negation. These conditions are associated with neurotransmitter imbalance, inflammatory responses, and oxidative stress in the brain. The composition of the present disclosure may regulate levels of serotonin, dopamine, and norepinephrine through nervonic acid, curcumin, and PS, suppress inflammatory cytokine release, scavenge free radicals, improve brain neurochemical environment, and relieve symptoms of depression and anxiety.

### BRIEF DESCRIPTION OF THE DRAWINGS

FIG. 1 shows the immobility time in the forced swim test for mice treated with the composition of Example 1 and various comparative examples in the depression improvement experiment;

FIG. 2 shows the oxidative stress level test results in mice treated with the composition of Example 1 and comparative examples in the depression improvement experiment;

FIG. 3 shows the oxidative stress level test results in the hippocampus of mice treated with the composition of Example 1 and comparative examples in the depression improvement experiment;

FIG. 4 shows the number of errors in descending the platform for mice treated with the composition of Example 1 and comparative examples in the memory enhancement experiment;

FIG. 5 shows the time ratio of exploring novel objects for mice treated with the composition of Example 1 and comparative examples in the memory enhancement experiment; and

FIG. 6 shows the test results of rotation counts induced in 6-OHDA Parkinson's disease model rats treated with the composition of Example 1 (labeled as 6-OHDA/ACP in the figure) and partial comparative examples (labeled as 6-OHDA/A for Comparative Example 5, 6-OHDA/Cu for Comparative Example 4, and 6-OHDA for the physiological saline control group) in the Parkinson's disease treatment experiment.

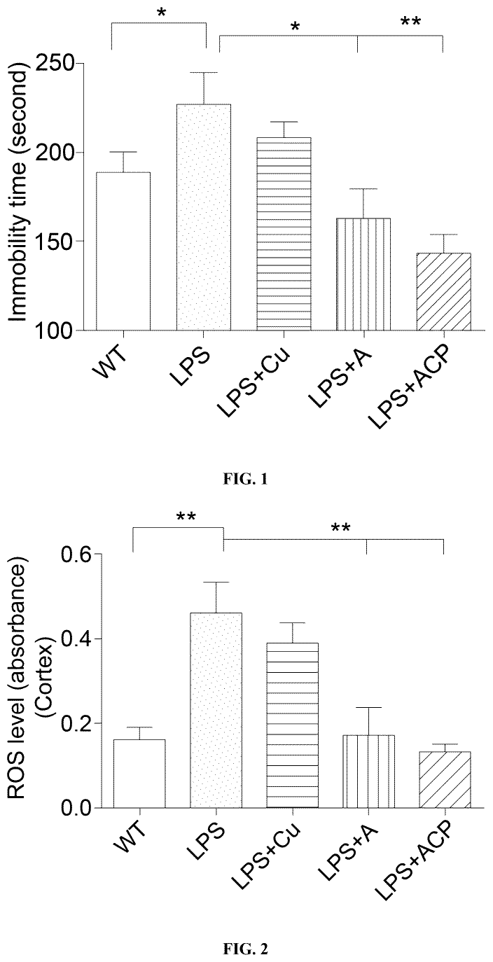

### DETAILED DESCRIPTION OF THE EMBODIMENTS

In order to better illustrate the objectives, technical solutions, and advantages of the present disclosure, the present disclosure will be further described below in conjunction with specific examples and accompanying drawings, but the examples do not limit the present application in any form. Unless otherwise specified, the reagents, methods, and devices used in the present application are conventional reagents, methods and devices in the prior art. Unless otherwise specified, the reagents and materials used in the present application are commercially available.

### Example 1

This example provided a neural repair composition, prepared according to the following formulation (by weight parts) and method:

1 part of turmeric powder, 45.5 parts of Acer truncatum bunge seed oil, 1 part of FAD, 0.5 part of α-tocopherol, and 0.5 part of vitamin B₆ were mixed and stirred uniformly to obtain the neural repair composition.

A preparation process of the turmeric powder included the following steps: dried turmeric root powder was added to 95% (v/v) ethanol. Ultrasonic extraction was conducted for 60 min to obtain an ethanol extract. The ethanol extract was vacuum-concentrated at 50° C. and 0.1 MPa to yield a concentrated solution. The concentrated solution was freeze-dried at −40° C. to obtain the turmeric powder.

The prepared turmeric powder included 5 wt % curcuminoid, with the following composition: C1 curcumin: 60 wt %, C2 demethoxycurcumin: 17 wt %, C3 bisdemethoxycurcumin: 1.5 wt %, and other types of curcuminoid as a balance.

### Example 2

This example provided a neural repair composition, prepared according to the following formulation (by weight parts) and method:

5 parts of turmeric powder, 228.75 parts of Acer truncatum bunge seed oil, 3 parts of FAD, 0.5 part of α-tocopherol, and 0.5 part of vitamin B₆ were mixed and stirred uniformly to obtain the neural repair composition. A preparation method of the turmeric powder was the same as that in Example 1.

### Example 3

This example provided a neural repair composition, prepared according to the following formulation (by weight parts) and method:

10 parts of turmeric powder, 459.5 parts of Acer truncatum bunge seed oil, 5 parts of FAD, 0.5 part of α-tocopherol, and 0.5 part of vitamin B₆ were mixed and stirred uniformly to obtain the neural repair composition. A preparation method of the turmeric powder was the same as that in Example 1.

### Example 4

This example provided a neural repair composition, prepared according to the following formulation (by weight parts) and method:

0.1 part of turmeric powder, 4.525 parts of Acer truncatum bunge seed oil, 0.1 part of FAD, 0.5 part of α-tocopherol, and 0.5 part of vitamin B₆ were mixed and stirred uniformly to obtain the neural repair composition. A preparation method of the turmeric powder was the same as that in Example 1.

### Example 5

This example provided a neural repair composition, which was prepared according to the method of Example 1, and was different from Example 1 in that the α-tocopherol and vitamin B₆ were not added.

### Comparative Example 1

This comparative example provided a neural repair composition containing only turmeric powder, prepared by the following method:

1 part of turmeric powder, 0.5 part of α-tocopherol, and 0.5 part of vitamin B₆ were mixed and stirred uniformly to obtain the neural repair composition. A preparation method of the turmeric powder was the same as that in Example 1.

### Comparative Example 2

This comparative example provided a neural repair composition containing only Acer truncatum bunge seed oil, prepared by the following method:

45.5 parts of Acer truncatum bunge seed oil, 0.5 part of α-tocopherol, and 0.5 part of vitamin B₆ were mixed and stirred uniformly to obtain the neural repair composition.

### Comparative Example 3

This comparative example provided a neural repair composition containing only FAD, prepared by the following method:

0.1 part of FAD, 0.5 part of α-tocopherol, and 0.5 part of vitamin B₆ were mixed and stirred uniformly to obtain the neural repair composition.

### Comparative Example 4

This comparative example provided a neural repair composition containing a combination of FAD and turmeric powder, compared to Example 1, prepared by the following method:

1 part of turmeric powder, 1 part of FAD, 0.5 part of α-tocopherol, and 0.5 part of vitamin B₆ were mixed and stirred uniformly to obtain the neural repair composition.

### Comparative Example 5

This comparative example provided a neural repair composition containing a combination of FAD and Acer truncatum bunge seed oil, compared to Example 1, prepared by the following method:

45.5 parts of Acer truncatum bunge seed oil, 0.1 part of FAD, 0.5 part of α-tocopherol, and 0.5 part of vitamin B₆ were mixed and stirred uniformly to obtain the neural repair composition.

### Application Example 1—Improvement of Depression by the Composition

The antidepressant effect of the composition was evaluated through the forced swim test in mice and measurement of oxidative stress levels in the cerebral cortex. Specific procedures were as follows:

Mice aged 8-9 weeks were selected as test subjects (10 mice per composition group for differential analysis and multiple comparisons; in differential analysis, * denoted p<0.05, ** p<0.01, *** p<0.001). The neural repair compositions prepared in the above examples and comparative examples were administered to mice via oral gavage. A positive control group treated with lipopolysaccharide (LPS) and a blank control group treated with physiological saline were included. From Days 1-5, mice were orally gavaged daily at the same time with 0.02 mL (normalized to body weight)/10 g of the compositions. On Days 3, 4, and 5, LPS (1 mg/kg) was intraperitoneally administered alongside the compositions. The positive control group differed by receiving only LPS (1 mg/kg) on Days 3, 4, and 5, while the blank control group received physiological saline throughout Days 1-5. On Day 6, behavioral tests (immobility time in the forced swim test, in s) and tissue sampling for oxidative stress assessment (reactive oxygen species absorption levels, expressed as absorbance) were conducted. The results are detailed in FIG. *to FIG. 2 and Tables 1-2.

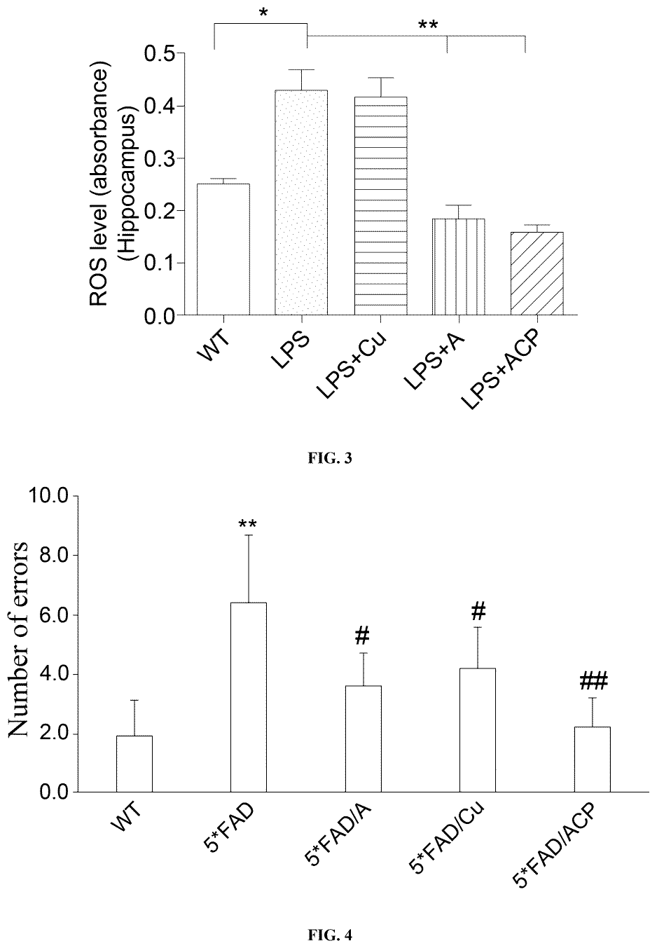

Additionally, oxidative stress levels in the hippocampus were tested following the same treatment protocol, with specific results shown in FIG. 3.

TABLE 1
Immobility time in the forced swim test of mice

| | | Immobility time | Significant |
|---|---|---|---|
| Composition | Treatment composition number | of mice (s) | difference |
| Example 1 | LPS + ACP | 145 ± 8 | ** |
| Example 2 | LPS + ACP | 143 ± 10 | ** |
| Example 3 | LPS + ACP | 165 ± 12 | ** |
| Example 4 | LPS + ACP | 159 ± 15 | ** |
| Example 5 | LPS + ACP | 150 ± 14 | ** |
| Comparative Example 1 | Turmeric powder | 210 ± 20 | * |
| Comparative Example 2 | Acer truncatum bunge seed oil | 216 ± 25 | * |
| Comparative Example 3 | PS (FAD) | 220 ± 29 | * |
| Comparative Example 4 | LPS + Cu | 212 ± 20 | * |
| Comparative Example 5 | LPS + A | 169 ± 23 | * |
| Positive control group | LPS | 230 ± 26 | * |
| Blank control group | WT | 188 ± 22 | * |

NOTE:
in the table: ACP represents the neural repair composition of the present disclosure, with active ingredients “curcumin + Acer truncatum bunge seed oil + PS”; LPS + A represents “Acer truncatum bunge seed oil + PS”; LPS + Cu represents “curcumin + PS”; WT represents the blank control group treated with physiological saline.

TABLE 2
Oxidative stress level test results in mice

| | Treatment | ROS absorption | Significant |
|---|---|---|---|
| Composition | composition number | level (absorbance) | difference |
| Example 1 | LPS + ACP | 0.09 ± 0.01 | ** |
| Example 2 | LPS + ACP | 0.08 ± 0.02 | ** |
| Example 3 | LPS + ACP | 0.10 ± 0.03 | ** |
| Example 4 | LPS + ACP | 0.11 ± 0.02 | ** |
| Example 5 | LPS + ACP | 0.11 ± 0.01 | ** |
| Comparative | Turmeric powder | 0.33 ± 0.08 | ** |
| Example 1 | | | |
| Comparative | Acer truncatum | 0.28 ± 0.09 | ** |
| Example 2 | bunge seed oil | | |
| Comparative | PS (FAD) | 0.32 ± 0.10 | ** |
| Example 3 | | | |
| Comparative | LPS + Cu | 0.35 ± 0.05 | ** |
| Example 4 | | | |
| Comparative | LPS + A | 0.14 ± 0.08 | ** |
| Example 5 | | | |
| Positive control | LPS | 0.47 ± 0.10 | ** |
| group | | | |
| Blank control | WT | 0.19 ± 0.06 | ** |
| group | | | |

NOTE:
in the table: ACP represented the neural repair composition of the present disclosure, with active ingredients “curcumin + Acer truncatum bunge seed oil + PS”; LPS + A represents “Acer truncatum bunge seed oil + PS”; LPS + Cu represents “curcumin + PS”; WT represents the blank control group treated with physiological saline.

### Application Example 2—Effects on Memory Enhancement

Experiment I: 6-month-old mice were treated with the compositions from the above examples and comparative examples, as well as physiological saline (as the blank control group), for 3 months. The dosage of Acer truncatum bunge seed oil in the compositions was standardized to 0.01 mL/g/d. After the treatment period, the step-down test was conducted to assess the number of errors in descending the platform. Each composition group included 10 mice for differential analysis. The test results are shown in Table 3 and FIG. 4.

TABLE 3
Number of errors in descending the platform by mice

| | Treatment | Number of errors | Significant |
|---|---|---|---|
| Composition | composition number | (times) | difference |
| Example 1 | ACP | 2.1 ± 0.9 | ** |
| Example 2 | ACP | 2.1 ± 0.6 | ** |
| Example 3 | ACP | 2.5 ± 0.9 | ** |
| Example 4 | ACP | 2.6 ± 1.0 | ** |
| Example 5 | ACP | 2.4 ± 0.8 | ** |
| Comparative Example 1 | Turmeric powder | 3.5 ± 1.2 | * |
| Comparative Example 2 | Acer truncatum bunge seed oil | 3.2 ± 1.3 | * |
| Comparative Example 3 | PS (FAD) | 3.8 ± 1.5 | * |
| Comparative Example 4 | Cu | 4.9 ± 1.6 | * |
| Comparative Example 5 | A | 4.0 ± 1.8 | * |
| Blank control group | WT | 6.3 ± 2.0 | ** |

### Experiment II: Time Ratio of Novel Object Exploration in Mice

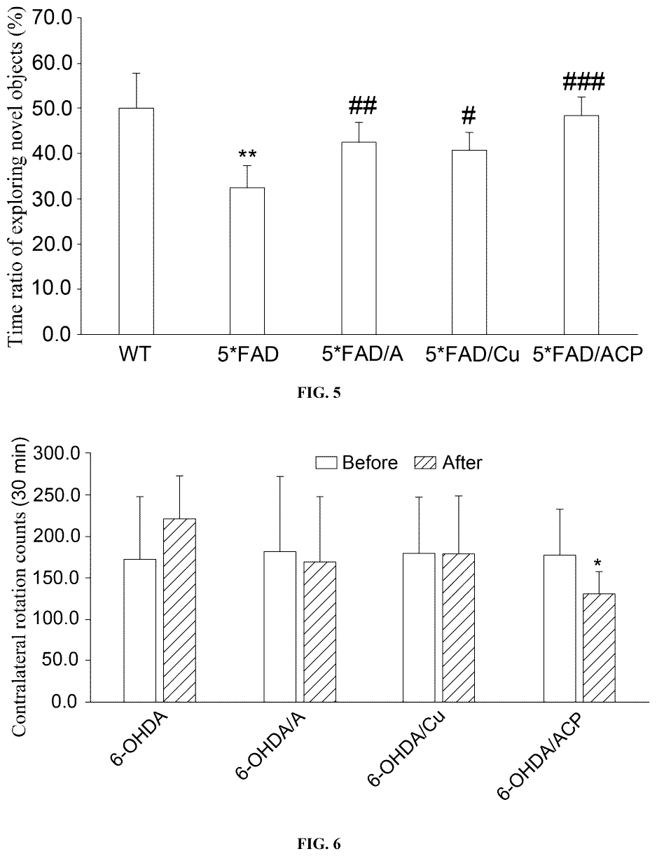

6-month-old mice were evenly divided into groups of 10. Each group was treated for 3 months with the compositions from the above examples and comparative examples, or physiological saline (as the blank control group), with the dosage of Acer truncatum bunge seed oil standardized to 0.01 mL/g/d. Novel object recognition was then assessed as follows: training phase: mice were exposed to two identical objects (A+A) in a chamber for 5 min. Testing phase: one familiar object was replaced with a novel object (A+B). Mice were reintroduced to the chamber to explore the objects for 5 min. Sessions were video-recorded with real-time tracking. Exploration was defined as the mouse directing its nose toward the object, sniffing/touching it, with the nose ≤2 cm from the object. Discrimination Index (DI) was calculated for each object as: (time exploring the novel object—time exploring the familiar object)/(time exploring the novel object+time exploring the familiar object)*100. The test results are shown in Table 4 and FIG. 5.

TABLE 4
Test results of time ratio for novel object exploration in mice

| | Treatment | | Significant |
|---|---|---|---|
| Composition | composition number | DI (%) | difference |
| Example 1 | ACP | 48.3 ± 3.2 | *** |
| Example 2 | ACP | 49.6 ± 3.6 | *** |
| Example 3 | ACP | 45.3 ± 3.3 | *** |
| Example 4 | ACP | 46.1 ± 2.8 | *** |
| Example 5 | ACP | 47.5 ± 3.0 | *** |
| Comparative Example 1 | Turmeric powder | 33.6 ± 2.4 | * |
| Comparative Example 2 | Acer truncatum bunge seed oil | 35.4 ± 1.8 | * |
| Comparative Example 3 | PS (FAD) | 32.5 ± 2.6 | * |
| Comparative Example 4 | Cu | 38.6 ± 3.1 | * |
| Comparative Example 5 | A | 40.3 ± 2.8 | *** |
| Blank control group | WT | 31.2 ± 4.5 | ** |

### Application Example 3—Application in Parkinson's Disease Treatment

Parkinson's disease model rats were induced by 3 weeks of 6-OHDA injections. These rats were then treated with the compositions from the above examples and comparative examples (physiological saline as the control group), with the dosage of Acer truncatum bunge seed oil standardized to 0.01 mL/g/d. After 2 weeks of treatment, rats were induced to rotate, and behavioral changes were observed in a quiet, spacious environment. Specific results are shown in FIG. 6.

From the above results, it was demonstrated that:

The neural repair composition prepared in the examples of the present disclosure exhibited significant efficacy in improving memory, alleviating depression, and treating Parkinson's disease.

Finally, it should be noted that the above examples are provided merely to describe the technical solutions of the present application, rather than to limit the protection scope of the present application. Although the present disclosure is described in detail with reference to preferred examples, a person of ordinary skill in the art should understand that modifications or equivalent replacements may be made to the technical solutions of the present disclosure without departing from the spirit and scope of the technical solutions of the present disclosure.

1-13. (canceled)

14. A neural repair composition, consisting of the following components in parts by weight:
0.1 part to 10 parts of curcumin or a turmeric powder, 4 parts to 460 parts of an Acer truncatum bunge seed oil, 0.1 part to 5 parts of flavin adenine dinucleotide and/or 0.01 part to 2 parts of an other excipient; wherein the curcumin comprises 2 wt % to 5 wt % of curcuminoid, the curcuminoid comprises 50 wt % to 60 wt % of C1 curcumin, 15 wt % to 17 wt % of C2 demethoxycurcumin, and 0.5 wt % to 1.5 wt % of C3 bisdemethoxycurcumin; and
the Acer truncatum bunge seed oil comprises 40 wt % to 50 wt % of linoleic acid, 15 wt % to 20 wt % of arachidonic acid, and 5 wt % to 8 wt % of nervonic acid.

15. The neural repair composition according to claim 14, comprising the following components in parts by weight:
1 part to 5 parts of the curcumin or the turmeric powder and 45 parts to 250 parts of the Acer truncatum bunge seed oil.

16. The neural repair composition according to claim 14, further comprising 0.01 part to 2 parts of a vitamin.

17. The neural repair composition according to claim 16, wherein the vitamin comprises 0.01 part to 1 part of vitamin E and 0.01 part to 1 part of vitamin B₆; and the vitamin E is selected from the group consisting of natural or synthetic α-tocopherol and a derivative thereof.

18. The neural repair composition according to claim 14, wherein the other excipient is one or a combination of two or more selected from the group consisting of a diluent, a preservative, and a suspending agent.

19. A method for preparing the neural repair composition according to claim 14, comprising the following steps:
Thoroughly mixing the components in the parts by weight to obtain the neural repair composition.

20. Use of the neural repair composition according to claim 14 in preparation of a health product for preventing or treating a neurodegenerative disease.

21. The use according to claim 20, wherein the neurodegenerative disease is selected from the group consisting of Alzheimer's disease, Parkinson's disease, amyotrophic lateral sclerosis (ALS), depression, and memory impairment.

22. The neural repair composition according to claim 16, comprising the following components in parts by weight:
1 part to 5 parts of the curcumin or the turmeric powder and 45 parts to 250 parts of the Acer truncatum bunge seed oil.

23. The method according to claim 19, wherein the neural repair composition comprises the following components in parts by weight:
1 part to 5 parts of the curcumin or the turmeric powder and 45 parts to 250 parts of the Acer truncatum bunge seed oil.

24. The method according to claim 19, wherein the neural repair composition further comprises 0.01 part to 2 parts of a vitamin.

25. The method according to claim 24, wherein the vitamin comprises 0.01 part to 1 part of vitamin E and 0.01 part to 1 part of vitamin B₆; and the vitamin E is selected from the group consisting of natural or synthetic α-tocopherol and a derivative thereof.

26. The method according to claim 19, wherein the other excipient is one or a combination of two or more selected from the group consisting of a diluent, a preservative, and a suspending agent.

27. The use according to claim 20, the neural repair composition comprises the following components in parts by weight:
1 part to 5 parts of the curcumin or the turmeric powder and 45 parts to 250 parts of the Acer truncatum bunge seed oil.

28. The use according to claim 20, the neural repair composition further comprises 0.01 part to 2 parts of a vitamin.

29. The use according to claim 28, wherein the vitamin comprises 0.01 part to 1 part of vitamin E and 0.01 part to 1 part of vitamin B₆; and the vitamin E is selected from the group consisting of natural or synthetic α-tocopherol and a derivative thereof.

30. The use according to claim 20, wherein the other excipient is one or a combination of two or more selected from the group consisting of a diluent, a preservative, and a suspending agent.
